(19) United States
(12) Patent Application Publication
(10) Pub. No.: US 2019/0298325 A1
(43) Pub. Date: Oct. 3, 2019

(54) BREAST SUBCUTANEOUS TISSUE SURGICAL INSTRUMENTS

(71) Applicant: Western New England University, Springfield, MA (US)

(72) Inventors: Steven Michael Schonholz, Warren, MA (US); Robert Treat Thompson Gettens, Longmeadow, MA (US); Jay Michael Branscomb, Hopkinton, MA (US); Karissa Kozlowski, West Springfield, MA (US); Steven Koss, JR., Tolland, CT (US)

(21) Appl. No.: 16/369,108

(22) Filed: Mar. 29, 2019

Related U.S. Application Data
(60) Provisional application No. 62/650,481, filed on Mar. 30, 2018.

(51) Int. Cl. A61B 17/02; A61M 29/00

(52) U.S. Cl. CPC A61B 17/02; A61B 2017/008; A61M 29/00

(57) ABSTRACT

A dilator configured for separating subcutaneous fat from glandular tissue includes a center section, a first end section extending from a first end of the center section, the first end section extending nonparallel to the center section, the first end section having a first diameter, and a second end section extending from a second end of the center section opposite the first end, the second end section extending nonparallel to the center section, the second end section having a second diameter different than the first diameter. A retractor includes a handle, a shaft extending from the handle, and a head located at a shaft end opposite the handle. The head includes a plurality of teeth, each tooth having a tooth end curving downwardly from a head outer surface, adjacent teeth spaced from one another by a tooth gap therebetween, the plurality of teeth having unequal tooth lengths.

### CROSS-REFERENCE TO RELATED APPLICATIONS

The present application is a Non-provisional Application of U.S. Provisional Application Ser. No. 62/650,481 filed on Mar. 30, 2018, the contents of which is incorporated by reference in its entirety.

### BACKGROUND

Exemplary embodiments pertain to the art of surgical instruments. In particular, the present disclosure relates to surgical instruments for separating subcutaneous fat from glandular breast tissue during a mastectomy procedure.

In treating breast cancer patients, the type of surgical procedure performed, if any, depends on the size and site of the tumor, characteristics of the tumor cells, the stage of the disease and the patient's choice. In breast conserving surgery, only the tumor and some surrounding tissue is removed. In another procedure, called a mastectomy, the entire breast and any surrounding infected areas are removed.

The current method to perform a mastectomy includes creating an incision around the breast and then separating the subcutaneous fat from the glandular breast tissue carefully using a scalpel while simultaneously using a retractor to hold the skin back. This procedure poses the risk of many complications because the space between the two tissues to be separated is thin, and the dissection extends down to the chest wall. During such a procedure, the skin or surrounding blood vessels may be punctured by the scalpel, which may lead to tissue necrosis or other post-operation complications.

An uncommon method to separate the two tissues is to use a dilator. After the incision is made and the skin is being retracted with the retractor, the surgeon uses a dilator to push between and loosen the connection between the subcutaneous fat and the glandular breast tissue, this causes a small hole to be created between the tissues. The surgeon will then continue to make small holes around the breast using the dilator until a clear plane can be seen between the two tissues. A scalpel is then used to complete the separation by “connecting the dots” the surgeon had previously made with the dilators.

This dilator method is a safer alternative to the standard scalpel method because it allows the surgeon to more clearly see where he or she will be cutting with the scalpel. Current available dilators, however, are designed for other uses and are not optimized for the anatomy of the breast tissue.

### BRIEF DESCRIPTION

In one embodiment, a dilator configured for separating subcutaneous fat from glandular tissue includes a center section, a first end section extending from a first end of the center section, the first end section extending nonparallel to the center section, the first end section having a first diameter, and a second end section extending from a second end of the center section opposite the first end, the second end section extending nonparallel to the center section, the second end section having a second diameter different than the first diameter.

Additionally or alternatively, in this or other embodiments the dilator is substantially C-shaped.

Additionally or alternatively, in this or other embodiments the first end section extends at a first end angle relative to the center section of between 8 degrees and 12 degrees.

Additionally or alternatively, in this or other embodiments the first end angle is between 9.5 degrees and 10.5 degrees.

Additionally or alternatively, in this or other embodiments the second end section extends at a second end angle relative to the center section, the second end angle equal to the first end angle.

Additionally or alternatively, in this or other embodiments the second end section extends at a second end angle relative to the center section of between 8 degrees and 12 degrees.

Additionally or alternatively, in this or other embodiments the second end angle is between 9.5 degrees and 10.5 degrees.

Additionally or alternatively, in this or other embodiments each of the first end section and the second end section have a spherical tip end.

Additionally or alternatively, in this or other embodiments the first end section, the second end section and the center section are formed together as a single unitary element.

In another embodiment, a retractor includes a handle, a shaft extending from the handle, and a head located at a shaft end opposite the handle. The head includes a plurality of teeth, each tooth having a tooth end curving downwardly from a head outer surface, adjacent teeth spaced from one another by a tooth gap therebetween, the plurality of teeth having unequal tooth lengths.

Additionally or alternatively, in this or other embodiments outermost teeth of the plurality of teeth have a first tooth length and innermost teeth of the plurality of teeth have a second tooth length less than the first tooth length.

Additionally or alternatively, in this or other embodiments one or more intermediate teeth are located between the outermost teeth and the innermost teeth, the intermediate teeth having a third tooth length greater than the second tooth length and less than the first tooth length.

Additionally or alternatively, in this or other embodiments each tooth has a tooth end, the plurality of tooth ends of the plurality of teeth defining one of a V-shaped or curvilinear profile across the retractor.

Additionally or alternatively, in this or other embodiments the tooth end curves downwardly through an angle of between 90 degrees and 180 degrees.

Additionally or alternatively, in this or other embodiments the angle is between 110 degrees and 150 degrees.

In yet another embodiment, a dilator set configured for separating subcutaneous fat from glandular tissue includes a plurality of dilators, each dilator including a center section, a first end section extending from a first end of the center section, the first end section extending nonparallel to the center section, the first end section having a first diameter, and a second end section extending from a second end of the center section opposite the first end, the second end section extending nonparallel to the center section, the second end section having a second diameter different than the first diameter. The first end diameter and the second diameter each dilator of the plurality of dilators are unique from the first diameter and the second diameter of each other dilator of the plurality of dilators.

Additionally or alternatively, in this or other embodiments the quantity of dilators in the dilator set is 5 dilators.

Additionally or alternatively, in this or other embodiments the first end diameters and the second end diameters of the plurality of dilators is in the range of 4.3 millimeters and 10.2 millimeters.

Additionally or alternatively, in this or other embodiments each dilator of the plurality of dilators is substantially C-shaped.

Additionally or alternatively, in this or other embodiments the first end section extends at a first end angle relative to the center section of between 8 degrees and 12 degrees.

### BRIEF DESCRIPTION OF THE DRAWINGS

The following descriptions should not be considered limiting in any way. With reference to the accompanying drawings, like elements are numbered alike:

FIG. 1 is a plan view of an embodiment of a dilator;

FIG. 2 is a partial plan view of an embodiment of a dilator;

FIG. 3 is a plan view of an embodiment of a dilator set;

FIG. 4 is a perspective view of an embodiment of a retractor;

FIG. 5 is a side view of an embodiment of a retractor;

FIG. 6 is a perspective view of another embodiment of a retractor; and

FIG. 7 is a perspective view of yet another embodiment of a retractor.

### DETAILED DESCRIPTION

A detailed description of one or more embodiments of the disclosed apparatus and method are presented herein by way of exemplification and not limitation with reference to the Figures.

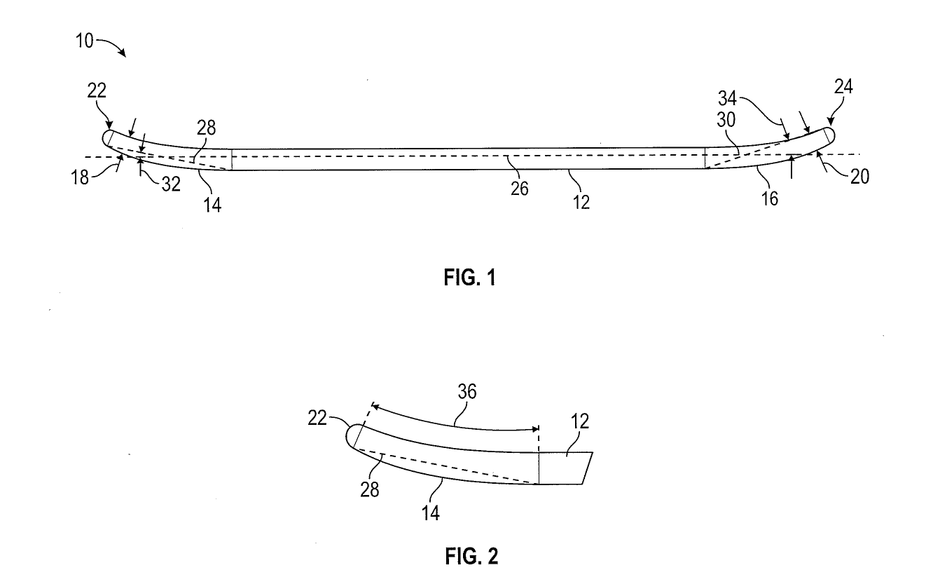

Referring to FIG. 1, illustrated is an embodiment of a dilator 10, configured for use in separating subcutaneous fat from glandular beast tissue during a mastectomy procedure. The dilator 10 is C-shaped, with a center section 12, a first end section 14 and a second end section 16, opposite the first end section 14. The first end section 14 and the second end section 16 each have a circular cross-section, with the first end section 14 having a first end diameter 18 and the second end section 16 similarly has a second end diameter 20. The first end diameter 18 is different from the second end diameter 20.

The first end section 14 includes a first end tip 22, and the second end section 16 similarly includes a second end tip 24. In some embodiments, the first end tip 22 and the second end tip 24 are formed as half spheres having radii of the first end section 14 and the second end section 16, respectively.

As stated above, the dilator 10 is C-shaped. The center section 12 extends along a center section axis 26, while the first end section 14 extends along a first section axis 28 and the second end section 16 extends along a second section axis 30. The first section axis 28 and the center section axis 26 define a first end angle 32, which in some embodiments is between 8 degrees and 12 degrees. In other embodiments, the first end angle 32 is between 9.5 degrees and 10.5 degrees. The second section axis 30 and the center section axis 26 define a second end angle 34, which in some embodiments is between 8 degrees and 12 degrees. In other embodiments, the first end angle 32 is between 9.5 degrees and 10.5 degrees.

In some embodiments, the first end section 14 and the second end section 16 extend substantially linearly from the center section 12 to their respective end tips 22, 24. In other embodiments, such as shown in FIG. 2, the first end section 14 and/or the second end section 16 extend curvilinearly to the end tips 22, 24. In some embodiments, the first end section 14 and/or the second end section 16 extend curvilinearly to the end tips 22, 24 through a tip arc angle 36 of between 20 degrees and 30 degrees. In other embodiments, the tip arc angle 36 is between 25 degrees and 27 degrees. While the first end section 14 is shown in FIG. 2, it is to be appreciated that the configuration is equally applicable to second end section 16.

Referring again to FIG. 1, the dilator 10 may be formed as a single, unitary component, or may, as shown in FIG. 1 be an assembly of two or more components. For example, the center section 12, the first end section 14 and the second end section 16 may be formed separately, and joined to form a dilator 10 by, for example, welding or other joining processes. Further, while the center section 12 is shown to be a single piece in FIG. 1, in some embodiments the center section 12 may be formed from multiple pieces assembled to form a center section 12 of a desired length. The dilator 10 is formed either from a metallic material, for example, 316L stainless steel or a polymeric material such as polyethylene.

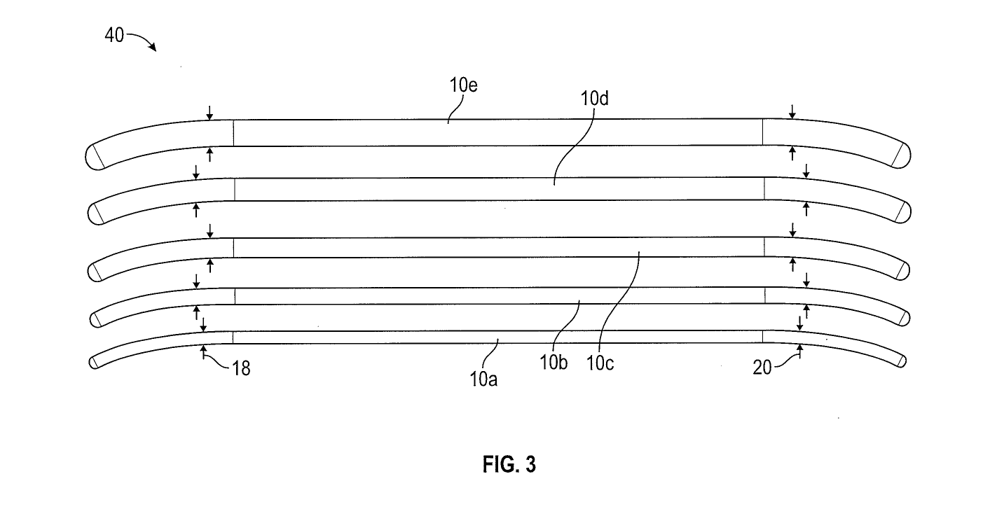

Referring now to FIG. 3, the dilators 10 may be manufactured and presented in dilator sets 40, which may have, for example, 5 dilators 10. In a dilator set 40, each dilator 10 has different first end diameters 18 and second end diameters 20, so that as the procedure progresses, different dilators 10 of different diameters may be utilized. For example, one dilator set may be configured such that a first dilator 10a has a first end diameter 18 of 4.3 millimeters and a second end diameter 20 of 4.6 millimeters; a second dilator 10b has a first end diameter 18 of 5.6 millimeters and a second end diameter 20 of 6.3 millimeters; a third dilator 10c has a first end diameter 18 of 7.0 millimeters and a second end diameter 20 of 7.6 millimeters; a fourth dilator 10d has a first end diameter 18 of 8.3 millimeters and a second end diameter 20 of 9.0 millimeters; and a fifth dilator 10e has a first end diameter 18 of 9.5 millimeters and a second end diameter 20 of 10.2 millimeters. It is to be appreciated that the above dimensions are merely exemplary, and that other first end diameters 18 and second end diameters 20 may be utilized. Further, while the dilator set 40 of FIG. 3 includes 5 dilators 10, other quantities of dilators may be utilized to form a dilator set 40.

The angled first end section 14 and second end section 16 of the dilator 10 better fits the anatomy of the breast tissue at which it is utilized, and the dual-end design with different diameters reduces the number of tools in the operating room.

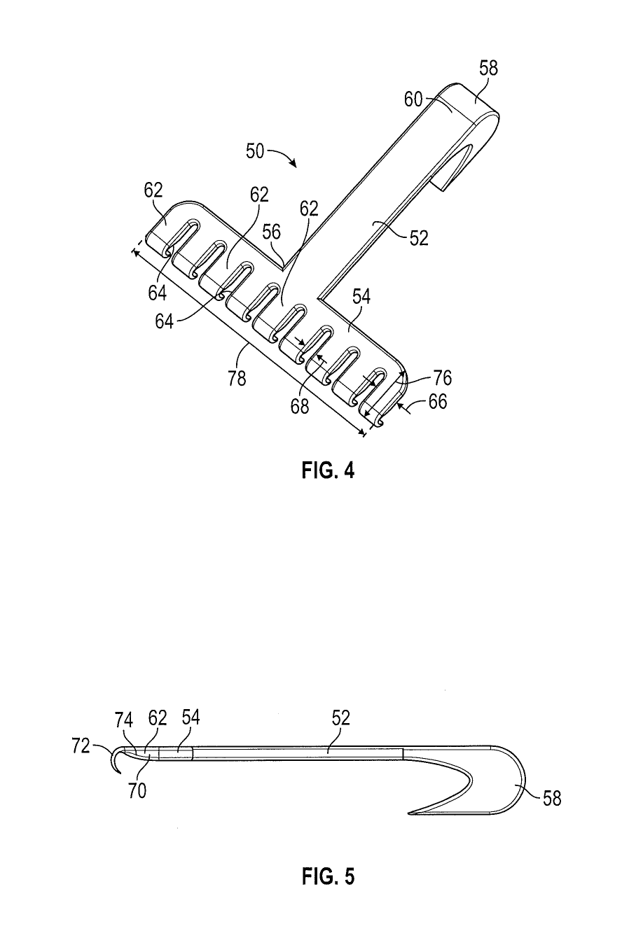

Referring to FIG. 4, an embodiment of a retractor 50 is illustrated. The retractor 50 includes a shaft 52 with a head 54 located at a first shaft end 56 of the shaft 52. A handle 58 is located at a second shaft end 60 opposite the first shaft end 56. In some embodiments, the retractor 50 is formed as a single unitary structure, while in other embodiments the handle 58, shaft 52 and head 54 are formed separately and assembled to form the retractor 50.

The head 54 includes a plurality of head teeth 62, separated from adjacent head teeth 62 by a plurality of tooth gaps 64. In some embodiments, the head teeth 62 are of the same tooth width 66, and the tooth gaps 64 are of the same gap width 68. It is to be appreciated, however, that in other embodiments the tooth width 66 and/or the tooth gaps 64 may vary. Further, in some embodiments, such as shown in FIG. 4, the tooth width 66 is substantially equal to the gap width 68. It is to be appreciated, however, that other configurations may be utilized in other embodiments.

Referring now to FIG. 5, each head tooth 62 has a tooth body 70 extending to a tooth end 72. The tooth body 70 is substantially flat, while the tooth end 72 curves downwardly from an outer surface 74 of the retractor 50. In some embodiments, the tooth end 72 curves downwardly through an angle of between 90 degrees and 180 degrees. In other embodiments, the tooth end 72 curves downwardly through an angle of between 110 degrees and 150 degrees. Referring again to FIG. 4, each head tooth 62 has an equal tooth length 76.

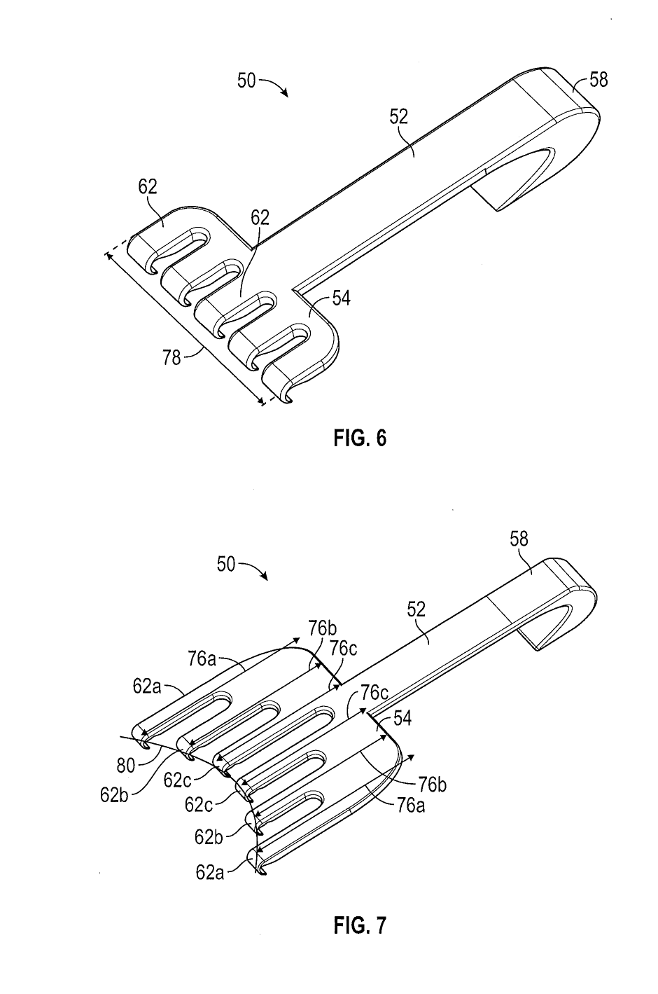

As shown in FIG. 4, in some embodiments, the retractor 50 has 9 head teeth 62 and may have a head width 78 of 6 inches. In another embodiment, as shown in FIG. 6, the retractor 50 has 5 head teeth 62 and a head width 78 of 3.5 inches. The head width 78 and number of head teeth 62 are configured based on, for example, the size of incision in which the retractor 50 is to be utilized.

Another embodiment of retractor 50 is shown in FIG. 7. In this embodiment, the retractor 50 has head teeth 62 which are staggered. For example, the outermost head teeth 62a have a first tooth length 76a, adjacent head teeth 62b have a second tooth length 76b such that the head teeth 62b are shorter than the head teeth 62a. Additionally innermost head teeth 62c have a third tooth length 76c shorter than the second tooth length 76b. Such a varying in the tooth lengths 76 results in a V-shaped or curved retractor end 80, defined by the positions of the tooth ends 72 of the head teeth 62. This configuration of retractor 50 with staggered head teeth 62 fits a curved region of the incision made during surgery.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A dilator configured for separating subcutaneous fat from glandular tissue, comprising:
a center section;
a first end section extending from a first end of the center section, the first end section extending nonparallel to the center section, the first end section having a first diameter; and
a second end section extending from a second end of the center section opposite the first end, the second end section extending nonparallel to the center section, the second end section having a second diameter different than the first diameter.

2. The dilator of claim 1, wherein the dilator is substantially C-shaped.

3. The dilator of claim 1, wherein the first end section extends at a first end angle relative to the center section of between 8 degrees and 12 degrees.

4. The dilator of claim 3, wherein the first end angle is between 9.5 degrees and 10.5 degrees.

5. The dilator of claim 3, wherein the second end section extends at a second end angle relative to the center section, the second end angle equal to the first end angle.

6. The dilator of claim 1, wherein the second end section extends at a second end angle relative to the center section of between 8 degrees and 12 degrees.

7. The dilator of claim 6, wherein the second end angle is between 9.5 degrees and 10.5 degrees.

8. The dilator of claim 1, wherein each of the first end section and the second end section have a spherical tip end.

9. The dilator of claim 1, wherein the first end section, the second end section and the center section are formed together as a single unitary element.

10. A retractor, comprising:
a handle;
a shaft extending from the handle; and
a head disposed at a shaft end opposite the handle, the head including:
a plurality of teeth, each tooth having a tooth end curving downwardly from a head outer surface, adjacent teeth spaced from one another by a tooth gap therebetween, the plurality of teeth having unequal tooth lengths.

11. The retractor of claim 10, wherein outermost teeth of the plurality of teeth have a first tooth length and innermost teeth of the plurality of teeth have a second tooth length less than the first tooth length.

12. The retractor of claim 11, further comprising one or more intermediate teeth disposed between the outermost teeth and the innermost teeth, the intermediate teeth having a third tooth length greater than the second tooth length and less than the first tooth length.

13. The retractor of claim 10, wherein each tooth has a tooth end, the plurality of tooth ends of the plurality of teeth defining one of a V-shaped or curvilinear profile across the retractor.

14. The retractor of claim 10, wherein the tooth end curves downwardly through an angle of between 90 degrees and 180 degrees.

15. The retractor of claim 14, wherein the angle is between 110 degrees and 150 degrees.

16. A dilator set configured for separating subcutaneous fat from glandular tissue, comprising:
a plurality of dilators, each dilator including:
a center section;
a first end section extending from a first end of the center section, the first end section extending nonparallel to the center section, the first end section having a first diameter; and
a second end section extending from a second end of the center section opposite the first end, the second end section extending nonparallel to the center section, the second end section having a second diameter different than the first diameter;
wherein the first end diameter and the second diameter each dilator of the plurality of dilators are unique from the first diameter and the second diameter of each other dilator of the plurality of dilators.

17. The dilator set of claim 16, wherein the quantity of dilators in the dilator set is 5 dilators.

18. The dilator set of claim 16, wherein the first end diameters and the second end diameters of the plurality of dilators is in the range of 4.3 millimeters and 10.2 millimeters.

19. The dilator set of claim 16, wherein each dilator of the plurality of dilators is substantially C-shaped.

20. The dilator set of claim 16, wherein the first end section extends at a first end angle relative to the center section of between 8 degrees and 12 degrees.
